(19) United States
(12) Patent Application Publication
(10) Pub. No.: US 2026/0013982 A1
(43) Pub. Date: Jan. 15, 2026

(54) INTRAOCULAR LENS USING MOIRE INTERFERENCE HYDROGEL

(71) Applicants: UNIVERSITY INDUSTRY FOUNDATION, YONSEI UNIVERSITY, Seoul (KR); KOREA INSTITUTE OF MACHINERY & MATERIALS, Daejeon (KR)

(72) Inventors: Won Gun KOH, Seoul (KR); Hyung Keun LEE, Seongnam-si (KR); Yong Woo JI, Seoul (KR); Se Min KIM, Seoul (KR); Chae Eun MOON, Seoul (KR); Gee Hong KIM, Daejeon (KR); Jae Jong LEE, Daejeon (KR)

(73) Assignees: UNIVERSITY INDUSTRY FOUNDATION, YONSEI UNIVERSITY, Seoul (KR); KOREA INSTITUTE OF MACHINERY & MATERIALS, Daejeon (KR)

(21) Appl. No.: 18/996,118

(22) PCT Filed: Jan. 18, 2023
(86) PCT No.: PCT/KR2023/000880

§ 371 Date: Jan. 17, 2025

(30) Foreign Application Priority Data
Jul. 20, 2022 (KR) 10-2022-0089281

(51) Int. Cl. A61F 2/14; A61L 27/18; A61L 27/52

(52) U.S. Cl. CPC A61F 2/145; A61L 27/18; A61L 27/52

(57) ABSTRACT

The present disclosure relates to an intraocular lens to which moire interference hydrogel is applied. The moire intraocular lens of the present disclosure can easily implement the presence and quantitative detection of target analytes by quantifying the volume change rate of the hydrogel through the moire signal.

### TECHNICAL FIELD

The present disclosure relates to an intraocular lens to which moiré interference hydrogel is applied.

### BACKGROUND ART

A hydrogel is a hydrophilic polymer with a three-dimensional stereoscopic structure that can easily include large amounts of moisture, genes, proteins, and cells. Since it has properties similar to living tissue, it has high biocompatibility, and thus has been widely used in biomedical fields, such as artificial organs, biosensors, drug delivery systems, cosmetics, and tissue engineering, for a long time.

Additionally, when the polymer chains constituting a hydrogel undergo structural or chemical changes or the degree of crosslinking of the polymer chains varies due to external control factors (e.g., temperature, pH, ionic strength, etc.), the chemical energy equilibrium formed between the hydrogel and water molecules undergoes a change, and causes the occurrence of inflow or outflow of water molecules, thereby expanding or reducing the volume of the hydrogel.

Hydrogels whose physical properties (e.g., volume, degree of crosslinking, strength, etc.) change along with the change in their structure in response to the external environment are called dynamic hydrogels, and are being actively used in the field of biotechnology such as studies relating to changes in the physiological behavior of cells and tissues, tissue engineering, drug delivery, and Bio-MEMS.

Dynamic hydrogels are manufactured by mixing and stirring monomers including organic molecules, proteins, etc., and then crosslinking and curing the resultant by ultraviolet ray irradiation or heating. As a representative example of using dynamic hydrogels as a polymer material for drug delivery, studies have been conducted on a system, in which when phenylboronic acid is used to react with glucose, the hydrogel into which phenylboronic acid is introduced expands and releases the drug or gene included therein. Among these studies, Patent Document 0001 discloses a hydrogel, in which a natural product with a cis-diol functional group and a polymer including phenylboronic acid are bound, and a drug delivery system using the same. However, even though a hydrogel is highly responsive to external stimuli, including high sensitivity not only to glucose but also to pH and active oxygen, it is difficult to actually manufacture a hydrogel with a volume change ratio of 70% or less, and thus, there are limitations in active utilization of dynamic hydrogels as biosensors.

In order to solve these problems, the inventors of the present disclosure, while conducting research, converted and amplified the changes in volume of a hydrogel into an optical signal so that when a change in the volume of the hydrogel was detected, it could easily be determined that the target material was present, and as a result of making efforts to apply and use the hydrogel to the human body, the inventors could complete the present disclosure.

### PRIOR ART DOCUMENT

### Patent Document

(Patent Document 0001) Patent Document 0001: Korean Patent No. 10-2075476.

### DETAILED DESCRIPTION OF INVENTION

### Technical Problems

The present disclosure relates to an intraocular lens to which a moire interference hydrogel is applied. An object of the present disclosure is to provide an intraocular lens that can quantify the volume change rate of a hydrogel through a moire signal and easily detect the presence and implement quantitative detection of a target analyte.

### Technical Solution

In order to achieve the object of the present disclosure as described above, there is provided a method for manufacturing a moire intraocular lens implantable into the eye, which includes:
a reference polymer hydrogel including a reference pattern;
a plurality of target analyte-sensitive polymer hydrogels to which each target analyte-specific probe is bound and which includes a comparison pattern; and
an intraocular lens to which the polymer hydrogel is bound.

Additionally, the present disclosure provides a method for manufacturing a moire intraocular lens implantable into the eye, which includes preparing an intraocular lens having a plurality of holes; and

linking a reference polymer hydrogel and a plurality of target analyte-sensitive polymer hydrogels to the intraocular lens.

### Effect of Invention

The moire intraocular lens of the present disclosure can easily detect the presence and

implement quantitative detection of a target analyte by quantifying the volume change rate of the hydrogel through the moire signal.

Additionally, unlike the use of fluorescent materials in conventional optical analysis methods, the present disclosure uses the transparent properties of a hydrogel to detect target analytes through the analysis of moire patterns; therefore, there is no need to add a labeling material, and detection can be accomplished by a simple method.

Additionally, the present disclosure provides the advantage in that it enables the detection of various types of markers by involving a compound with a desired biochemical functional group in the polymer chain polymerization process.

Additionally, the present disclosure enables detection of extremely small amounts of target analytes by amplifying the detection signal of the target analyte through changes in the moire pattern, and it is easy to control the effect of amplifying the detection signal by controlling the concentration of the target analyte-specific probe.

### BRIEF DESCRIPTION OF THE DRAWING

FIG. 1 is a schematic diagram showing the operation principle of a sensing module using moire.

FIG. 2 is a drawing of moire patterns for monitoring the volume change of a responsive polymer hydrogel (a schematic diagram in which (a) shows an embodiment of a moire pattern consisting of two sets of parallel lines, where one set is tilted at a certain angle with respect to the other set, and (b) shows the moire patterns formed by overlapping the reference grid and the hydrogel grid at different volumes).

FIG. 3 is a schematic diagram for manufacturing an MIOL according to an embodiment of the present disclosure.

FIG. 4 is a diagram showing a pig eye into which an MIOL is inserted according to an embodiment of the present disclosure.

FIG. 5 shows images of an MIOL according to an embodiment of the present disclosure.

FIG. 6 is an image of a pig eye into which an MIOL has been inserted according to an embodiment of the present disclosure.

FIG. 7 shows images and a graph illustrating the changes in moire signals upon injection of a BDNF solution (222 nM) into a pig eye implanted with an MIOL according to an embodiment of the present disclosure (left: optical images of moire patterns, right: a quantitative analysis graph illustrating the changes in pitch size from reference and BDNF-sensitive hydrogels).

FIG. 8 shows images illustrating the process of inserting an MIOL into a rabbit eye through cataract surgery according to an embodiment of the present disclosure.

FIG. 9 shows images confirming the inflammatory response 60 days after inserting an MIOL according to an embodiment of the present disclosure into a rabbit eye.

### BEST MODE FOR CARRYING OUT THE INVENTION

Hereinafter, the constitutions of the present disclosure will be described in detail. In particular, unless otherwise defined, all technical and scientific terms have meanings commonly understood by those skilled in the art in the technical field to which this invention pertains, and the terms used in the description of the present disclosure are only intended to effectively describe specific embodiments but are not intended to limit the present disclosure.

Additionally, in the following description, the descriptions of known effects and constitutions that may unnecessarily obscure the gist of the present disclosure are omitted. In the specification below, the units used without special mention are based on weight, and for example, units of % or ratio mean wt % or weight ratio.

Additionally, as used herein in the specification, the singular forms “a,” “an,” and “the” may be intended to include the plural forms as well, unless the context clearly states otherwise.

The present disclosure provides a moire intraocular lens that is implantable into the eye and a method of manufacturing the same.

The moire intraocular lens implantable into the eye according to the present disclosure may include a reference polymer hydrogel including a reference pattern; a plurality of target analyte-sensitive polymer hydrogels to which each target analyte-specific probe is bound and which includes a comparison pattern; and an intraocular lens to which the polymer hydrogel is bound.

A hydrogel is a high molecular weight polymer in the form of a crosslinked network consisting of one or more monomers, and has a high moisture content, thereby allowing various biomolecules to be immobilized within the hydrogel while maintaining their structures and activities. The moire intraocular lens of the present disclosure can detect target analytes through a change in the volume of the hydrogel according to the binding to the target analyte within the eye by linking the hydrogel fixed to the target analyte-specific probe to the intraocular lens. Furthermore, it is possible to perform quantitative detection of target analytes with high sensitivity by amplifying the volume change in the hydrogel through moire signals.

In the present disclosure, the target analyte-specific probe may be one which is fixed to the surface of the hydrogel by crosslinking with the polymer chain inside the target analyte-sensitive polymer hydrogel.

In the present disclosure, the reference pattern and the comparison pattern may overlap each other to form a moire pattern.

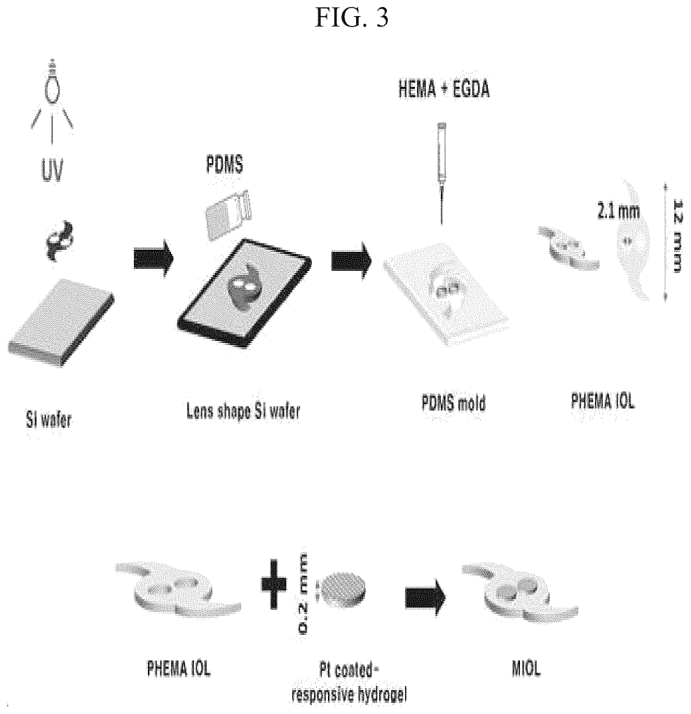

In an example of the present disclosure, a reference polymer hydrogel and a target analyte-specific probe are bound, and a target analyte-sensitive polymer hydrogel including a pattern is bound to an intraocular lens, and a schematic diagram of the manufacturing method is shown in FIG. 3.

There may be one or more target analyte-sensitive polymer hydrogels, and the target analyte-sensitive polymer hydrogels may each bind to a different target analyte-specific probe. For example, the moire intraocular lens may be in a form, in which a first target analyte-sensitive polymer hydrogel (where one reference polymer hydrogel and an anti-brain derived neurotrophic factor (anti-BDNF) are bound), and a second target analyte-sensitive polymer hydrogel to which an anti-platelet derived growth factor (anti-PDGF) is bound are bound to an intraocular lens, but is not limited thereto, and a target analyte-sensitive polymer hydrogel, to which each different target analyte-specific probe is linked, may be further added.

The intraocular lens to which the polymer hydrogel is attached may be manufactured directly as shown in FIG. 3, but this is only an embodiment, and a commercially-available intraocular lens may also be used. When a commercially-available intraocular lens is used, the hydrogel can be inserted into a location that does not block the optical part of the intraocular lens (e.g., the haptic part).

In the present disclosure, the target analyte-specific probe may be a biomolecule that recognizes an analyte and may be one or more selected from the group consisting of an aptamer, a peptide, an enzyme, a hormone receptor, an antibody, an antigen, and a cell.

The target analyte-specific probe may be introduced with a predetermined functional group in order to be fixed on the hydrogel, and in the present disclosure, the target analyte-specific probe may be one introduced with an acrylate functional group.

As the target analyte-specific probe is included in the polymer chain constituting the hydrogel, it responds to the target analyte and enables the formation of a bond between the target analyte-specific probe and the target analyte. One target analyte molecule can interact with two or more target analyte-specific probes to thereby induce a change in the volume of the hydrogel according to the binding of the target analyte. Accordingly, the target analyte, which is bound to two or more target analyte-specific probes, can induce volume shrinkage of the hydrogel by forming physical crosslinking points.

In the present disclosure, the reference polymer hydrogel and the target analyte-sensitive polymer hydrogel may be based on the same or different polymers, and the polymer is not limited as long as the linear polymer constituting the hydrogel is a water-soluble polymer. Specifically, the polymer may be any one or more selected from the group consisting of polyethylene glycol (PEG), polyvinyl alcohol (PVA), polyvinylpyrrolidone (PVP), polyacrylamide, polyacrylic acid, and a copolymer thereof, alginate, agarose, cellulose, gelatin, collagen, hyaluronic acid, and chitosan, but is not particularly limited as long as it is a synthetic polymer or natural polymer that can fix biomolecules.

In the present disclosure, the reference pattern and the comparison pattern may overlap each other to form a moire pattern, and the shape of the pattern may be a fishbone pattern, a ladder pattern, or a parallel grid pattern. Specifically, the parallel grid pattern refers to a pattern in which a plurality of parallel straight lines with a certain thickness are arranged at regular intervals. The reference pattern and the comparison pattern may each be formed at regular intervals, preferably in the range of 5 nm to 100 nm, more preferably at intervals of 10 nm to 80 nm, and most preferably at intervals of 15 nm to 60 nm. In the above-mentioned pattern, the line width may be 0.5 nm to 50 nm, preferably 1 nm to 20 nm, but is not particularly limited thereto. It is preferable for easy measurement of changes in the moire pattern if the reference pattern and the comparison pattern are manufactured to have similar line widths and spacing.

In the present disclosure, the intraocular lens may be based on poly(2-hydroxyethyl methacrylate) (PHEMA), polymethyl methacrylate (PMMA), poly(lactic acid-glycolic acid) (PLGA), and polyvinylpyrrolidone, or silicone hydrogel, but is not particularly limited as long as it is a material capable of manufacturing intraocular lenses commonly known in the art.

In a preferred embodiment of the present disclosure, the hydrogel may be further coated with platinum. Specifically, platinum may be coated on the hydrogel to a thickness of nm to 50 nm, preferably 20 nm to 30 nm, and through this, a clear pattern image may be obtained by controlling the refractive index of the hydrogel surface. When the hydrogel is coated excessively, the image may become dark, and thus, it is possible to easily measure the moire signal by coating with a thickness in the above-mentioned range.

The reference polymer hydrogel including a reference pattern may be manufactured in the same manner as described above, but when it is projected onto a comparison pattern and used to form a moire pattern, it is not necessary for the target analyte-specific probe to be in a fixed form.

Additionally, the present disclosure provides a method for manufacturing a moire intraocular lens implantable into the eye, which includes preparing an intraocular lens having a plurality of holes; and

linking a reference polymer hydrogel and a plurality of target analyte-sensitive polymer hydrogels to the intraocular lens.

The steps for preparing a target analyte-sensitive polymer hydrogel including a comparative pattern, to which a target analyte-specific probe according to the present disclosure is bound, are specifically as follows.

As a non-limiting example, a biomolecule, such as an antibody capable of forming a specific binding to a target analyte, is treated with acrylic acid N-hydroxysuccinimide to prepare a biomolecule into which an acrylate functional group is introduced. The biomolecule, into which an acrylate functional group is introduced, is mixed with a polymer precursor solution; a crosslinking agent, an initiator, and a catalyst are added thereto; and then a hydrogel is prepared through UV photopolymerization. In particular, when the polymerization solution including the precursor solution, crosslinking agent, initiator, and catalyst for polymerization are added to a mold having a certain pattern and polymerized, it is possible to obtain a hydrogel with a desired pattern. Since the patterning method can be performed by conventional techniques known in the art, detailed description will be omitted.

In a preferred embodiment of the present disclosure, the polymer precursor solution may include a pore-inducing material (porogen). The porogen may be specifically, for example, inorganic oxides including silica, titania, zirconia, etc., a derivative thereof, or a mixture thereof. The porogen is not crosslinked during the polymerization process and but is removed later, thereby forming pores in the hydrogel and obtaining a porous hydrogel.

As used herein, the term “moire” pattern of the present disclosure refers to an interference fringe generated when two or more periodic patterns overlap, and from the academic view, the moire pattern may be defined as a unique pattern of low frequencies generated by the beat phenomenon when several grids with similar periods overlap.

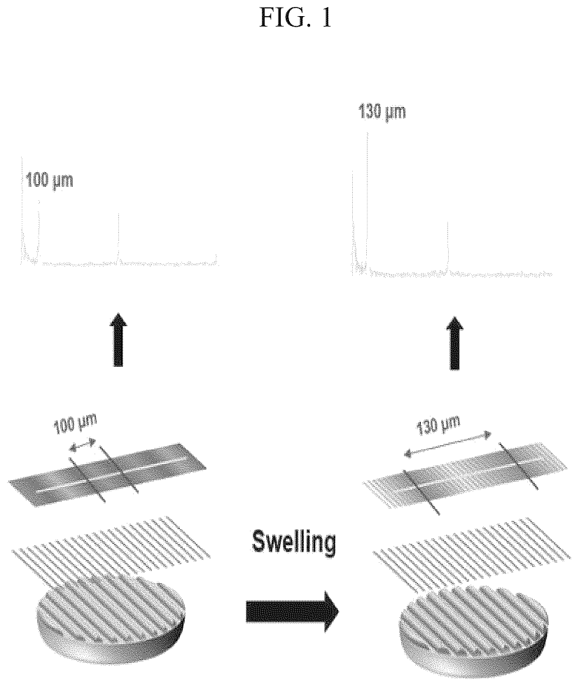

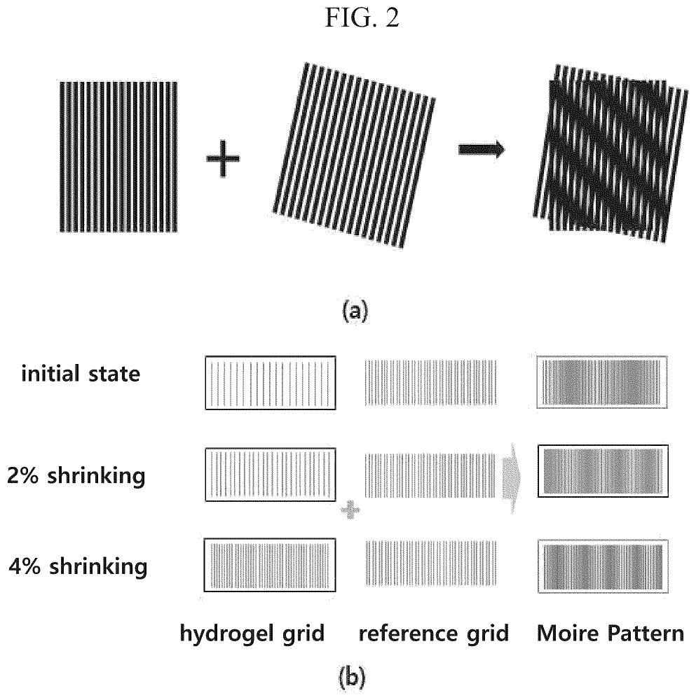

To describe with reference to FIGS. 1 and 2, when light is irradiated onto a reference polymer hydrogel including a reference pattern while a light source is disposed, a shadow of the reference pattern may overlap on the surface of the comparison pattern to form a moire pattern, and this is defined as the initial moire signal.

When a target analyte is brought into contact with the target analyte-sensitive polymer hydrogel, a specific bond is formed between the target analyte-specific probe, which has been fixed by crosslinking with the polymer chain inside the hydrogel, and the target analyte. By the binding between the probe and the analyte, the hydrogel shrinks, and a slight pitch change occurs in the pattern arrangement according to the change in the volume of the hydrogel. Accordingly, the initial moire signal changes, and through this change, it is possible to easily determine the presence or absence of the target material, and perform quantitative detection by analyzing the intensity of the moire signal.

In particular, preferably, two or more probes fixed on the internal chain of the hydrogel along the pattern can form a specific bond with one target analyte, and the amplification of the moire signal can be maximized by increasing the degree of shrinkage of the hydrogel.

Hereinafter, the present disclosure will be described in more detail through preparation examples, experimental examples, and examples. However, the following examples are only a reference for describing the present disclosure in detail, and the present disclosure is not limited thereto, and may be implemented in various forms.

### Preparation Example 1. Preparation of Target Analyte-Sensitive Polymer Hydrogel with Comparison Pattern Formed

A brain-derived neurotrophic factor (BDNF)-sensitive polymer hydrogel was prepared as follows. Anti-BDNF was dissolved in 100 μL of a PBS buffer solution to a concentration of 1 mg/mL, and 33.3 μL of 2.22 mmol anti-BDNF was reacted with acrylic acid-NHS at 25° C. for 3 hours to generate acylated-BDNF, which is a modified antibody. In particular, the ratio between anti-BDNF and acrylic acid-NHS was 1:6. In order to remove unreacted materials, dialysis was performed for one day using a 2000 MWCO dialysis kit. After dialysis, 0.211 mmol acrylamide and 6.486 μmol N′, and N′-methylenebisacrylamide (MBAA) were dissolved in the modified antibody solution, and the volume was adjusted to 97 μL by adding the PBS buffer solution. 2.5 μL of 10 wt % ammonium persulfate (APS) and 0.5 μL of tetramethylethylenediamine (TEMED) were added thereto, mixed for 1 second, and formed into a pattern with a silicone mold having a thickness of 200 μm onto a line-patterned silicon wafer with a pitch size of 16 μm. After covering the resultant using a cover glass, it was polymerized at 25° C. to prepare a hydrogel on which anti-BDNF was fixed. The thus-prepared hydrogel was washed with purified water, dried completely, and coated with a thickness of 20-30 nM of platinum for 250 seconds to thereby complete a BDNF-sensitive hydrogel.

### Preparation Example 2. Preparation of Reference Polymer Hydrogel

0.211 mmol acrylamide and 6.486 μmol N′, N′-methylenebisacrylamide (MBAA) were dissolved in 97 μm of a PBS buffer solution. 2.5 μm of 10 wt % ammonium persulfate (APS) and 0.5 μL of tetramethylethylenediamine (TEMED) were added thereto, mixed for 1 second, and formed into a pattern with a silicone mold having a thickness of 200 μm onto a line-patterned silicon wafer with a pitch size of 16 μm. After covering the resultant using a cover glass, it was polymerized at 25° C. to prepare a hydrogel. The thus-prepared hydrogel was washed with purified water, dried completely, and coated with a thickness of 20-30 nM of platinum for 250 seconds to thereby prepare a reference polymer hydrogel.

### Preparation Example 3. Preparation of Moire Intraocular Lens (MIOL)

The hydrogels prepared in Preparation Examples 1 and 2 were inserted into a PHEMA-based intraocular lens (IOL) support manufactured using a PDMS mold. The series of processes for manufacturing an MIOL are schematically shown in FIG. 3.

A PDMS mold was prepared using a conventional replica molding process. A Si master including a replica pattern of the PDMS mold was prepared using SU-8 50 through spin coating, pre-baking, UV exposure, post-baking, and development processes, and the mold was designed to have two holes in the IOL. The resulting IOL-shaped PDMS mold was filled with 80 μL of a precursor solution consisting of 77.2 μL of HEMA, 2 μL of EGDA, and 0.8 μL of HOMPP, and was cured by exposing it to UV for 280 seconds (365 nm, 5300 mW/cm², EXFO OmniCure Series 1000, UV spot lamp, Mississauga, Ontario, Canada). A disc-shaped reference and target material-sensitive polymer hydrogels with a diameter of 2 mm were inserted into each hole of the IOL, and a moire intraocular lens equipped with the reference and target material (BDNF)-sensitive polymer hydrogels was completed through protein binding.

### Experimental Example 1. Detection of Target Materials in In Vitro Environment

The moire intraocular lens prepared in Preparation Example 3 above was implanted into a porcine eye and tested in vitro.

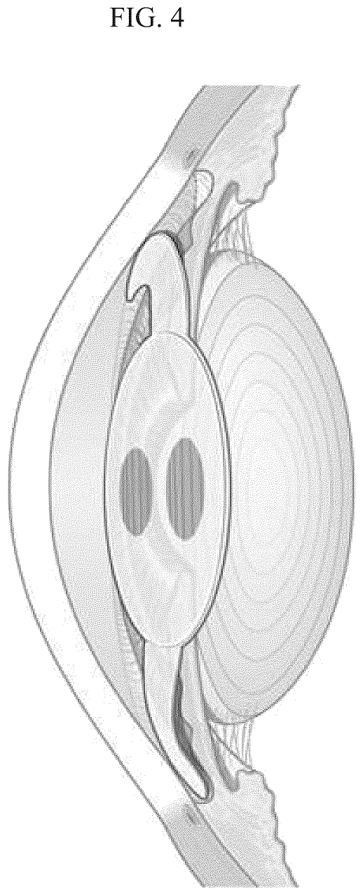

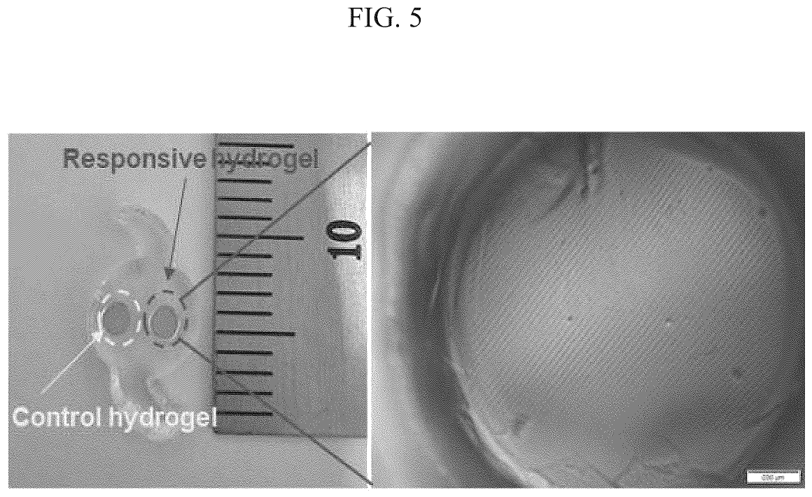

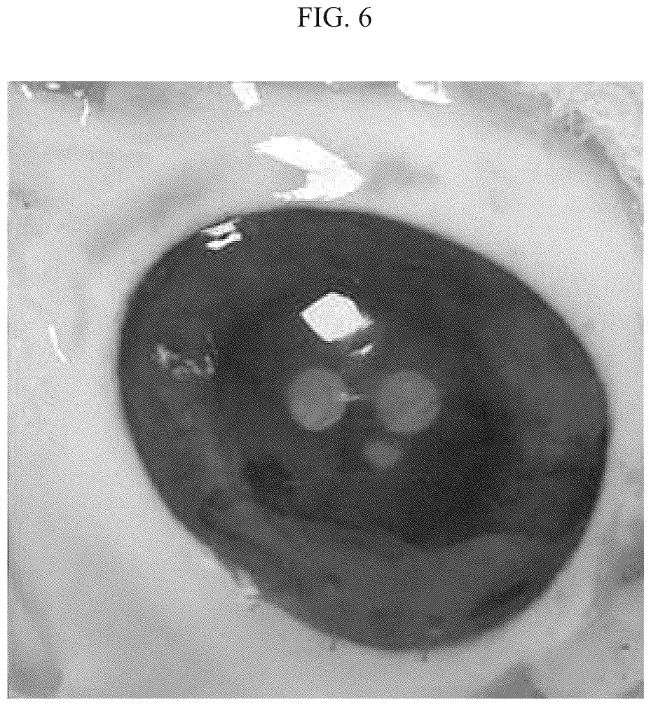

A moire intraocular lens including reference and target material-sensitive polymer hydrogels was implanted between the cornea and lens of a pig eye, as shown in FIG. 4. FIG. 5 shows an MIOL loaded with reference and BDNF-sensitive polymer hydrogels, in which microgroove patterns prepared through an embodiment of the present disclosure can be observed; and FIG. 6 shows that the implanted MIOL is well fixed on the pig eye and could be seen with the naked eye.

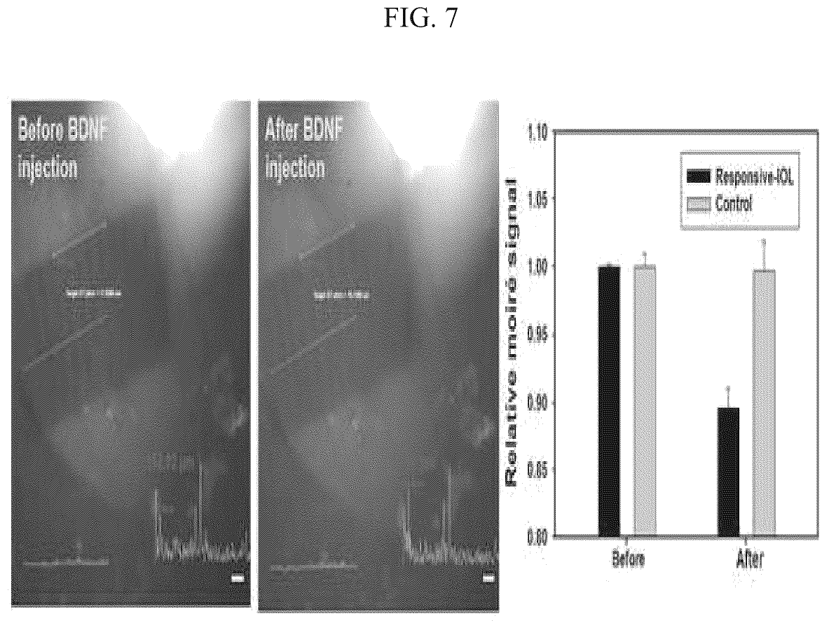

As shown in FIG. 7, when 700 μL of a 222 nM BDNF solution in PBS was injected into a pig eye, and the moire signal was decreased from 152.92 μm to 133.80 μm in the BDNF-sensitive polymer hydrogel; however, there was no change in the reference polymer hydrogel. These results confirm that the target protein (BDNF) can diffuse into the hydrogel grid inserted into the IOL and bind to anti-BDNF, thereby being capable of causing shrinkage of the hydrogel in an in vitro environment. The initial moire signal (152.92 μm) was different from the value obtained in the in vitro experiment (93.09 μm) because the pitch size of the reference grid overlapped on the hydrogel grid was reduced by the curvature of the eye.

### Experimental Example 2. Confirmation of Biocompatibility in in Vivo Environment

The MIOL prepared in Preparation Example 3 above was implanted into the eyes of a live rabbit and a biocompatibility test was performed thereon.

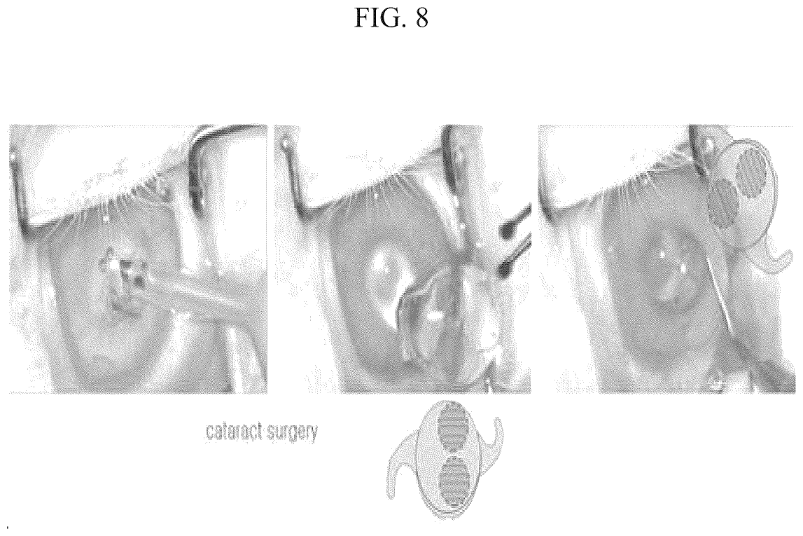

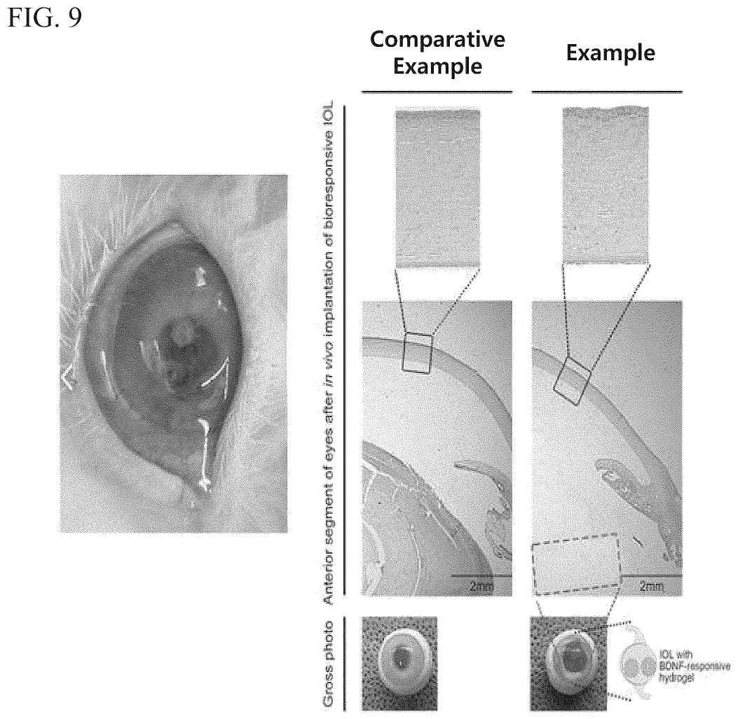

MIOL was inserted through cataract surgery as shown in FIG. 8. 60 days after the insertion, rabbit eyeballs were extracted and the presence/absence of inflammation was confirmed through H&E staining. Referring to FIG. 9, it was confirmed that no inflammatory reaction was observed in both in Comparative Example (without the insertion of an MIOL) and Example (with the insertion of an MIOL).

1. A moire intraocular lens implantable into the eye, comprising:
a reference polymer hydrogel comprising a reference pattern;
a plurality of target analyte-sensitive polymer hydrogels to which each target analyte-specific probe is bound and which comprises a comparison pattern; and
an intraocular lens to which the polymer hydrogel is bound.

2. The moire intraocular lens of claim 1, wherein the target analyte-specific probe is crosslinked with a polymer chain inside the target analyte-sensitive polymer hydrogel and fixed to the surface of the hydrogel.

3. The moire intraocular lens of claim 1, wherein the reference pattern is overlapped with a comparison pattern to form a moire pattern.

4. The moire intraocular lens of claim 1, wherein the reference pattern and the comparison pattern are each in the form of a parallel grid with regular intervals of 5 nm to 100 nm.

5. The moire intraocular lens of claim 1, wherein the probe is one or more selected from the group consisting of an aptamer, a peptide, an enzyme, a hormone receptor, an antibody, an antigen, and a cell.

6. The moire intraocular lens of claim 1, wherein the polymer hydrogel is based on one or more selected from the group consisting of polyethylene glycol (PEG), polyvinyl alcohol (PVA), polyvinylpyrrolidone (PVP), polyacrylamide, polyacrylic acid, and a copolymer thereof, alginate, agarose, cellulose, gelatin, collagen, hyaluronic acid, and chitosan.

7. The moire intraocular lens of claim 1, wherein the target analyte-specific probe is introduced with an acrylate functional group.

8. The moire intraocular lens of claim 1, wherein the intraocular lens is based on poly(2-hydroxyethyl methacrylate) (PHEMA), polymethyl methacrylate (PMMA), poly(lactic acid-glycolic acid) (PLGA), and polyvinylpyrrolidone (PVP), polyvinylacetate (PVA), or silicone hydrogel.

9. A method of manufacturing a moire intraocular lens implantable into the eye, comprising:
preparing an intraocular lens having a plurality of holes; and
linking a reference polymer hydrogel and a plurality of target analyte-sensitive polymer hydrogels to the intraocular lens.
